(19) United States
(12) Patent Application Publication
(10) Pub. No.: US 2007/0031367 A1
(43) Pub. Date: Feb. 8, 2007

(54) Composition and method for neck skin firming

(76) Inventors: Vera Brown, Los Angeles, CA (US); Yorum Fishman, Riverside, CA (US)

Correspondence Address: CHRISTIE, PARKER & HALE, LLP, PO BOX 7068, PASADENA, CA, 91109-7068, US

(21) Appl. No.: 11/519,731

(22) Filed: Sep. 11, 2006

Related U.S. Application Data
(63) Continuation-in-part of application No. 10/932,690, filed on Sep. 1, 2004, now abandoned.

(51) Int. Cl. A61K 8/97; A61K 36/06; A61K 36/82; A61K 36/48; A61K 36/81

(52) U.S. Cl. 424/74; 424/760; 424/757; 424/780; 424/729; 424/195.15

(57) ABSTRACT

A topical composition and method for its use for reducing sagging or wrinkles in the neck area of human skin. The composition includes safe and effective amounts of a mild topical astringent (such as Camphor) for skin tightening, a topical antispasmodic (such as benzaldehyde or benzyl cinnamate) for skin smoothing, and a topical skin irritant to increase localized skin circulation. The composition can also optionally include palmitoyl oligiopeptides to stimulate production of collagen and elastin in skin, a mushroom or plant leaf ferment extract to increase the cell turnover rate skin, and to increase hydration, skin firmness and elimination of fine facial lines, a cocao ferment extract to inhibit lipid peroxidation, inhibit excess ornithine decarboxylase activity, and stimulate cutaneous blood flow to reduce lines and wrinkles, and a Lactobacillus lysate extract to stimulate collagen production. These ingredients are mixed in a dermatologically acceptable carrier for topical application.

### CROSS-REFERENCE TO RELATED APPLICATIONS

This application is a continuation-in-part of U.S. application Ser. No. 10/932,690, entitled “COMPOUND FOR NECK SKIM FIRMING” filed on Sep. 1, 2004.

### SUMMARY OF THE INVENTION

This invention is related to the field of skin firming and tightening compositions, and more particularly to a composition and method particularly well-suited to firming and tightening wrinkled and sagging skin of a person's neck.

It is natural that as a person ages, his or her skin lose some of its natural elasticity, smoothness and “glow” that is more commonly associated with younger individuals. Besides genetic predispositions to certain skin types, extrinsic conditions such as exposure to ultraviolet radiation (e.g. too much sun or tanning lamps), pollution, wind, heat, low humidity, and harsh chemicals can speed up this process. Lifestyle choices can also negatively affect the skin, with smoking, poor nutrition, excess alcohol consumption and mental and physical stress accelerating the aging process. Intrinsic factors such as natural aging and illness also take their toll. Besides wrinkles and sagging, other forms of roughness can become evident, including increased pore size, flaking and skin lines. While this breakdown is part of the natural aging process, it can also result from excessive squinting, frowning and even smiling can even contribute to this.

Physiologically, the extrinsic or intrinsic stretched and wrinkled skin can result as the underlying collagen and elastin fibers in the skin begin to break down and wear out. Collagen is a triple helix formed by three extended protein chains that wrap around one another. Elastin is polypeptide chains that are cross-linked together to form rubber-like, elastic fibers.

Not being satisfied to let nature (and sometimes bad personal choices and personal care) take its course, several approaches to reduce wrinkles and skin sagging have been developed.

A radical approach to tightening of the skin includes cosmetic surgery, where a surgeon will cut away excess skin, and sometime remove or add fat to effectuate a tightening of skin. However, this is highly invasive, relatively expensive, can leaves scars, and can sometimes leave a patient with a stretched appearance.

Recently, lasers have been joined in the battle against wrinkles, and lasers are now being used for laser skin peel. In this technique, laser light is used to peel away the skin to cause rejuvenated skin growth. This technique is expensive, invasive and can be painful. Caustic chemicals, such as phenol, can also be used to peel away the top layers of skin to stimulate the grown of new skin that will hopefully be smoother and less wrinkled. Ask with laser peels, peels can be traumatic and are generally painful, and may result in skin splotchiness.

Another invasive technique is dermabrasion or skin sanding. This technique involves sanding the surface of the skin, including wrinkles and scars. The physician uses a high-speed motorized diamond sanding wheel, a wire brush, or sandpaper to grind away the surface of the skin. Dermabrasion is most effective on the flat areas of the face, upper lip, forehead, and chin, but it is dangerous and difficult to use dermabrasion around the eyes and on some other areas of the face because of natural curves in the face and varying thickness of skin. The treatments soften the sharp edges of surface irregularities, giving the skin a smoother appearance.

Collagen injections for wrinkles have been one of the top ten cosmetic procedures performed in the United States since they were approved by the FDA in 1981. Injectable collagen is a natural substance derived from purified bovine (cow) collagen and is used to replace the collagen the skin loses over time. The injectable collagen is placed just beneath the skin where the body hopefully accepts it as its own. By supplementing a patient's skin's own collagen with injectable collagen, some wrinkles and scars can be smoothed out. The appearance of deeper frown lines and nasal labial furrows can also be decreased. While collagen injections may be relatively safe and effective non-surgical wrinkle treatment, some patients are allergic to bovine collagen. Also, this bovine collagen tends to absorbed by the body with time, so repeated injections are required.

In April of 2002, the Food and Drug Administration approved Botulinum Toxin Type A (Botox Cosmetic) for temporarily improving the appearance of moderate to severe frown lines between the eyebrows (glabellar lines). Botulinum Toxin Type A is a protein produced by the bacterium Clostridium botulinum. When used in medical settings as an injectable form of sterile, purified botulinum toxin, small doses of the toxin are injected into the affected muscles and block the release of the chemical acetylcholine that would otherwise signal the muscle to contract. The toxin thus paralyzes or weakens the injected muscle and gives the skin a temporarily smoother appearance. The affects of Botox are only temporary, and the procedure must be repeated for maximum effectiveness.

There are also many personal care products currently available to consumers directed to improving the health and/or appearance of the skin. Some of these products are designed to delay, minimize or even eliminate skin wrinkling and other histological changes (e.g. sagging skin) that are associated with the aging of skin and environmental damage to skin.

Some common approaches that formulators of wrinkle creams take are to include moisturizers and hydrators (since dryness can contribute to wrinkles) and to supplement the lost collagen and fibrin in the skin.

However, despite numerous promises, there are still few products that effectively reduce wrinkles of one particular trouble spot, the skin of the neck. The physiology and morphology of the skin can vary dramatically depending on which part of the body it covers. For example, the skin around the eyes is thinner than skin of other areas of the body and is more subject to crowfoot wrinkles, while the skin of the forehead tends to develop longer, more longitudinal furrows. The skin of the neck is somewhat unique in that it generally receives poor circulation than other types of skin, and as a result, many people developed flabby, loose necks as they age.

There accordingly remains a need for a safe and effective composition and method of use that are particularly well-suited to firming and tightening the wrinkled and sagging skin of a person's neck, and which can do so safely, quickly and painlessly.

The invention provides a topical containing a mixture of active ingredients and method for their use designed to work together to reduce wrinkles and sagging skin, particularly in the neck area. The composition includes:
a topical astringent such as camphor (or other compositions like thymol, peppermint oil, menthol and/or tea tree oil);
a composition such as benzyl cinnamate (or other composition such as benzyl benzoate, benzyl nicotinate and/or benzyl alcohol), which has an antispasmodic action and can promote the growth of epithelial (skin tissue) cells; and
an ingredient that stimulates circulation in the skin such as a capsaicinoids like Capsaicin. Helpful optional ingredients are palmitoyl pentapeptide-3, palmitoyl tetrapeptide-3, and/or acetyl hexapeptide-3 which have been shown to stimulate production of collagen and elastin in skin. Other optional active ingredients can include mushroom ferment extracts, to increase the cell turnover rate skin, and to increase hydration, skin firmness and elimination of fine facial lines, Cocoa ferment extract to inhibit lipid peroxidation, inhibit excess ornithine decarboxylase activity, and stimulate cutaneous blood flow to reduce lines and wrinkles, and Lactobacillus lysate extracts such as Lactobacillus/Trifolium Pratense (clover) flower ferment extract, and Lactobacillius/Camellia Sinensis leaf ferment extract to stimulate collagen production in the skin.

These ingredients are mixed into a lotion or cream base to deliver the active ingredients to the skin of the neck.

### DETAILED DESCRIPTION OF THE INVENTION

The invention discloses a unique topical composition for reducing neck wrinkles and sagging neck skin.

The invention provides a composition containing a mixture of active ingredients designed to work together to reduce neck wrinkles and sagging neck skin, including a topical astringent which tends to draw together or constrict skin tissue, such as Camphor (or other compositions like thymol, peppermint oil, menthol and/or tea tree oil), a composition such as benzyl cinnamate (and/or benzaldehyde, benzyl nicotinate and benzyl alcohol), which has an antispasmodic action and also increases localized circulation; and an ingredient to irritant the skin and stimulate circulation in the skin, such as a capsaicinoids like Capsaicin. Other helpful optional ingredients are palmitoyl pentapeptide-3, palmitoyl tetrapeptide-3, and acetyl hexapeptide-3 which have been shown to stimulate production of collagen and elastin in skin. Other optional active ingredients can include mushroom ferment extracts to increase the cell turnover rate in the skin, and to increase hydration, skin firmness and elimination of fine facial lines, Cocoa ferment extract to inhibit lipid peroxidation, inhibit excess ornithine decarboxylase activity, and stimulate cutaneous blood flow to reduce lines and wrinkles, and Lactobacillus lysate extract such as Lactobacillus/Trifolium Pratense(clover) flower ferment extract, and Lactobacillius/Camellia Sinensis leaf ferment extract to stimulate collagen production in the skin.

The active ingredients are put in a dermatologically acceptable lotion or cream base carrier for topical application to the skin to be treated.

The composition of one such exemplary composition is set forth in Table 1 below, with the active ingredients being listed first and the inactive ingredients being the ingredients of one possible dermatologically acceptable carrier. Other carrier formulations can be used in lieu of that listed below.

TABLE 1
ONE EXEMPLARY COMPOSITION

| | Function | % w/w |
|---|---|---|
| Active ingredients | | |
| Camphor | A mild skin astringent that | 1.60 (0.10 |
| | causes skin tightening and | to 10.00 |
| | increases localized | |
| | circulation | |
| Benzyl cinnamate | An antispasmodic that can | 0.80 (0.10 |
| | promote the growth of | to 10.00 |
| | epithelial (skin tissue) | |
| | cells and also increases | |
| | localized circulation | |
| Capsicum Frutescens Fruit | A skin irritant that | 0.40 (0.10 |
| Extract | stimulates localized | to 5.0) |
| | circulation in skin | |
| Palmitoyl Pentapeptide-3 | Increases production of | 3.00 (0.01 |
| (Matricyl ™) | collagen | to 10.0) |
| Water & butyl glycol & | To increase the cell | 0.10 (.05 |
| Lactobacillus/Lentinus (Shitake | turnover rate skin, | to 10.00) |
| mushroom) ferment extract & | and to increase hydration, | |
| lactobacillus/Ganoderma | skin firmness and | |
| Lucidum (Reishi/Mannentaken | elimination of fine | |
| mushroom) ferment extract | facial lines | |
| Water & Lactobacillus/ | To inhibit lipid | 0.10 (.05 |
| Theobroma Cacoa | peroxidation, inhibit excess | to 10.00) |
| (Cocoa) ferment extract | ornithine decarboxylase | |
| | activity, and stimulate | |
| | cutaneous blood flow to | |
| | reduce lines and wrinkles | |
| Water & Lactobacillus Lysate | To stimulate collagen | 0.10 (.05 |
| Extract | production in the skin | to 10.00) |
| Dermatologically | | |
| acceptable carrier | | |
| ingredients | | |
| Water (Aqua Purificata) Purified | Solvent | 52.0020 |
| Mineral oil | Lubricant & emollient | 15.10 |
| Sorbitan sesquioleate | Emulsifier & moisturizer | 7.33 |
| Petrolatum | emollients & moisturizer | 6.60 |
| Ceresin | A thickening wax | 6.23 |
| Lanolin | Softener | 3.67 |
| Fragrance (parfum) | For fragrance | 2.36 |
| Methylparaben | Preservative | 0.25 |
| Propylparaben | Preservative | 0.10 |
| FD&C Certified Red 33 | Coloring (if desired) | 0.00056 |

TABLE 2
ANOTHER EXEMPLARY COMPOSITION

| | Function | % w/w |
|---|---|---|
| Active ingredients | | |
| Camphor | A mild skin astringent that | 1.600 |
| | causes skin tightening and | (0.10 to |
| | increases localized | 10.00 |
| | circulation | |
| Benzaldehyde | An antispasmodic that can | 2.360 |
| | promote the growth of | (0.10 to |
| | epithelial (skin tissue) | 10.00 |
| | cells and also increases | |
| | localized circulation | |
| Capsicum Frutescens Fruit | A skin irritant that | 0.400 |
| Extract | stimulates localized | (0.10 to |
| | circulation in skin | 5.0) |
| Glycerin and butylene glycol | Increases production of | 0.500 |
| and water carbomer | collagen | (0.01 to |
| and palmitoyl | | 10.0) |
| oligiopeptide and palmitoyl | | |
| tetrapeptide-3 (Matricyl ™) | | |
| Lactobacillus/Trifolium | To increase the cell turnover | 0.010 |
| Pratense (clover) flower | rate skin, and to increase | (.010 to |
| ferment extract & | hydration, skin firmness and | 10.000) |
| Lactobacillus/Theobroma | elimination of fine facial | |
| Cacoa (Cocoa) | lines | |
| ferment extract &) | | |
| Lactobacillus/Camellia | | |
| Senensis | | |
| leaf ferment extract | | |
| Water & Lactobacillus/ | To inhibit lipid | 0.10 (.05 |
| Theobroma Cacoa (Cocoa) | peroxidation, inhibit excess | to 10.000) |
| ferment extract | ornithine decarboxylase | |
| | activity, and stimulate | |
| | cutaneous blood flow to | |
| | reduce lines and wrinkles | |
| Hydrolyzed collagen | To provide collagen to the | 0.010 |
| | skin | (0.001 to |
| | | 10.000) |
| Dermatologically | | |
| acceptable carrier | | |
| ingredients | | |
| Water (Aqua Purificata) | Solvent | 52.002 |
| Purified | | |
| Mineral oil | Lubricant & emollient | 15.100 |
| Sorbitan sesquioleate | Emulsifier & moisturizer | 7.330 |
| Petrolatum | emollients & moisturizer | 6.600 |
| Ceresin | A thickening wax | 6.230 |
| Lanolin | Softener | 3.670 |
| Fragrance (parfum) | For fragrance | 2.36 |
| Methylparaben | Preservative | 0.250 |
| Propylparaben | Preservative | 0.100 |
| FD&C Certified Red 33 | Coloring (if desired) | 0.028 |

Below is description of various active compositions used in the formulation of the exemplary compositions of the invention of Tables 1 and 2. In lieu of the carrier described in Tables 1 and 2, other dermatologically acceptable carrier ingredients can be used as a carrier.

Camphor is present in the exemplary composition of the invention as a mild skin astringent. This mild skin astringent tends to draw together or constrict the skin, thereby reduce sagging skin and fine lines. In the exemplary embodiment of Tables 1 and 2, it is present at 1.600% w/w, but can be present in the range of 0.100 to 10.000% w/w. Besides camphor, other ingredients that can be used in the composition include thymol, peppermint oil, menthol and/or tea tree oil, just to name a few of the possible compositions.

Benzyl Cinnamate may be included in the composition of the invention for its antispasmodic action (which tends to smooth the skin), which helps to in the skins maintenance processes. This ingredient tends to smooth and tighten the skin and thereby reduce sagging skin and fine lines. In the particular composition of Table 1, it is present at 0.8% w/w, but can be present in the range of 0.10 to 10.00% w/w. Benzyl Cinnamate has a pleasant cinnamon-like aroma, which it imparts to the composition. Other ingredients that can be similarly used including Benzaldehyde (having an almond-like smell and present in at 2.360% w/w/ in the composition of Table 2 the range of 0.10 to 10.00% w/w), Benzyl nicotinate, and benzyl alcohol.

The composition of the invention utilizes Capsaicin as an ingredient in order to stimulate circulation in the skin of the neck and lower chin. The Capsicum Frutescens Fruit Extract used in the exemplary embodiment of the invention shown in Tables 1 and 2 contain capsaicin in the range of 60,000 to 100,000 Scoville Units, and can be sourced from Bio Botanica, Inc., of Hauppauge, N.Y. Other products that can be used in lieu of Capsaicinoids include oil of black pepper and benzyl nicotinate, both of which increase localized circulation in the skin by causing skin irritation.

Capsaicin, also known as N-canillyl-8-methyl-6-(E)-noneamide, is the most pungent of the group of compositions called Capsaicinoids (a crystalline alkaloid) that can be isolated from chili peppers. It is sparingly soluble in water, but very soluble in fats, oils and alcohol. The minor Capsaicinoids include Nordihydrocapsaicin [Dihydrocapsaicin with a (CH₂)₅ instead of (CH₂)₆], Homocapsaicin [Capsaicin with a (CH₂)₅ instead of (CH₂)₄, and Homodihydrocapsaicin [Dihydrocapsaicin with a (CH₂)₇ instead of (CH₂)₆]. The chemical structures of these compositions are shown below:

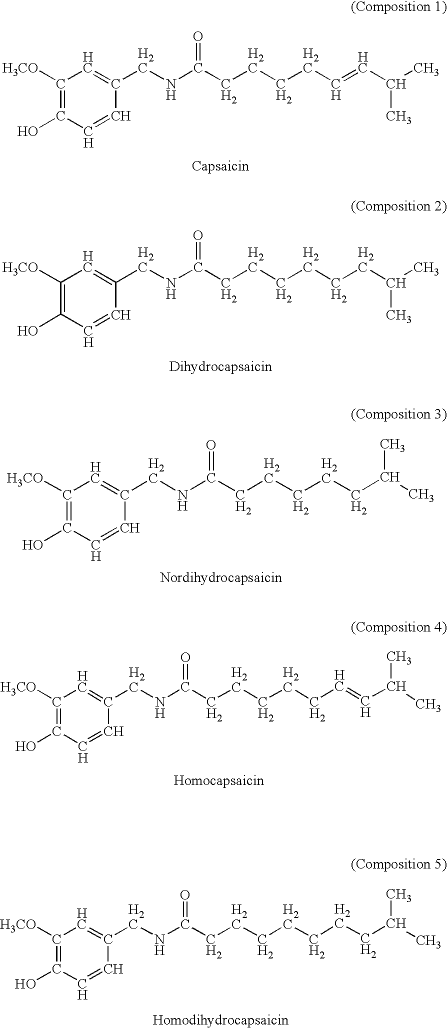

Table 3 a comparison chart showing the Scoville Units of various varieties of peppers. As shown in the figure, pure Capsaicin has about 16,000,000 Scoville units.

TABLE 3
Scoville Units per Pepper Variety

| | Scoville Units | Pepper Variety |
|---|---|---|
| | 16,000,000 | Pure Capsaicin |
| 10 | 100000-350000 | Habanero, Scotch Bonnet |
| 9 | 50000-100000 | Santake, Thai |
| 8 | 30000-50000 | Cayenne, Tabasco, Piquin |
| 7 | 15000-30000 | Serrano, Chile de Arbol |
| 6 | 5000-15000 | Yellow Wax, Serrano |
| 5 | 2500-5000 | Jalapeno (up to 10,000), Miasol |
| 4 | 1500-2500 | Sandia, Cascabel |
| 3 | 1000-1500 | Ancho (Poblano), Pasilla, |
| | | Espanola |
| 2 | 500-1000 | Anaheim, New Mexico |
| 1 | 100-500 | Mexi-bells, Cherries |
| 0 | 0-100 | Mild Bells, Sweet Bananas, |
| | | Pimento |

Although peppers can vary from pod to pod, plant to plant, variety to variety, and even season to season, there is some consistency. A Scoville Unit is a measurement of capsaicin level in a particular pepper or variety.

Also beneficially included is a mixture of Palmitoyl Oligiopeptides that contains Palmitoyl pentapeptide-3, and Palmitoyl tetrapeptide-3, in a mixture of glycerin, butylene glycol and carbomer, offered under the trademark name Matricyl™ by Sederma, S. A., a member of the Croda International Group, of Parsippany, N.J. Palmitoyl pentapeptide-3 is a synthetic pentapeptide (Palmitoyl-Lys-Thr-Thr-Lys-Ser) that mimics a fragment of Procollagen Type I by stimulating the matrix molecules (Collagen I, III and IV and Fibronectin), and it and other Palmitoyl Oligiopeptides including Palmitoyl tetrapeptide-3 have been shown in some studies to reduce the appearance of wrinkles. This active ingredient can preferably be included at a 3.00% w/w in the composition of Table 1 and about 0.500% w/w/ in the composition of Table 2, but can be eliminated or have its percentage changed to the range of changed to the range of 0.010 to 10.000% w/w if desired.

Water & butyl glycol & Lactobacillus/Lentinus (Shitake mushroom) ferment extract & lactobacillus/Ganoderma Lucidum(Reishi/Mannentaken mushroom) ferment extract, such as the ACB Mushroom Extract SM, Code Number 20353, offered by the company Active Concepts, of South Plainfield, N.J., increases the cell turnover rate, enhances skin hydration, skin firmness and helps reduce fine facial lines and thus is a helpful optional ingredient in the composition. In the exemplary composition shown in Table 1, it is included at a 0.10% w/w, but can be eliminated, or presented at a different concentration range (e.g. from 0.05% to as high 10%.)

Lactobacillus/Trifolium Pratense (clover) flower ferment extract & Lactobacillus/Theobroma Cacoa (Cocoa) ferment extract & Lactobacillus/Camellia Senensis leaf ferment extract such as the ACB CelluPlex Code Number 20206 offered by the company Active Concepts, of South Plainfield, N.J., is rich in phytoestrogen compositions. This composition can increase the cell turnover rate skin, and to increase hydration, skin firmness and elimination of fine facial lines. In the exemplary composition shown in Table 2, it is included at a 0.010% w/w, but can be eliminated, or presented at a different concentration range (e.g. from 0.001% to as high 10%) if desired.

Water & Lactobacillus/Theobroma Cacoa (Cocoa) ferment extract, such as the ACB Cocoa Bioferment, Code Number 20331, offered by the company Active Concepts, of South Plainfield, N.J., is rich in xanthine alkaloids, flavonols, and related oligomers. This composition can reduce platelet aggregation, inhibit lipid peroxidation, inhibit excess Ornithine Decarboxylase activity, and stimulates cutaneous blood flow to reduce lines and wrinkles and is useful for skin firming. In the exemplary composition shown in Table 1, it is included at a 0.10% w/w, but can be eliminated, or presented at a different concentration range (e.g. from 0.05% to as high 10%) if desired.

Lastly, a water & Lactobacillus Lysate Extract, ACB Yogurt DRF, Code Number 20220, offered by the company Active Concepts, of South Plainfield, N.J., has platelet-derived growth factor (PDGF), insulin-like growth factor (IGF-I, IGF-II), as well as transforming growth factor (TGF-a), which growth factors stimulate fibroblast proliferation as well as the production of extra-cellular proteins such as collagen. In the exemplary composition shown in Table 1, it is included at a 0.10% w/w, but can be eliminated, or presented at a different concentration range (e.g. from 0.05% to as high 5.00%.)

In summary, the active ingredients of the invention includes topical agents that increase localized circulation in the skin, such as a Capsaicinoids, camphor and/or benzyl cinnamate or benzaldehyde. Additional optional active ingredients include ingredients such as Palmitoyl Oligiopeptides, Palmitoyl pentapeptide-3 and Palmitoyl tretrapeptide-3 to cause increased localized production of the matrix molecules (Collagen I, III and IV and/or Fibronectin in the skin. Hydrolyzed collagen can also be included. Lactobacillus Lysate Extract can optionally be included to stimulate collagen production in the skin. This can include Lactobacillus/Trifolium Pratense (clover) flower ferment extract & Lactobacillus/Theobroma Cacoa (Cocoa) ferment extract & Lactobacillus/Camellia Senensis leaf ferment extract. Other optional ingredients include ingredients to increase the cell turnover rate skin, and to increase hydration, skin firmness and elimination of fine facial lines, such as shitake and Reishi mushroom extracts. Optional ingredients such as Lactobacillus/Theobroma Cocoa extras can inhibit lipid peroxidation, inhibit excess ornithine decarboxylase activity, and stimulate cutaneous blood flow to reduce lines and wrinkles.

The above note compositions are to be used by topical application to the skin of the neck of a human on an as-needed basis to improve the condition of the skin of the neck by reducing the appearance of presence of wrinkles, sags and fine line and to tighten up the skin of the neck. This use may be infrequently, or once or as often as several times a day.

It will be recognized by those skilled in the art that various modifications may be made to the illustrated and other embodiments of the invention described above, without departing from the broad inventive scope thereof. It will be understood therefore that the invention is not limited to the particular embodiments or arrangements disclosed, but is rather intended to cover any changes, adaptations or modifications which are within the scope and spirit of the invention as defined by the appended claims.

What is claimed is:

1. A topical composition for reducing sagging or wrinkles in the skin of the neck of humans, comprising:
a safe and effective amount of a composition that stimulates increased localized circulation in the skin comprising a Capsaicinoid in a range of 60,000 to 100,000 Scoville Units in a weight percent range of 0.01 to 5.0 wt/wt;
a safe and effective amount of an antispasmodic composition comprising benzaldehyde or benzyl cinnamate in a weight percent range of 0.010 to 10.000 wt/wt;
a safe and effective amount of skin astringent comprising camphor in a weight percent range of 0.01 to 5.0 wt/wt; and
a dermatologically acceptable carrier.

2. The topical composition of claim 1, wherein the Capsaicinoid comprises Capsicum Frutescens Fruit Extract.

3. The topical composition of claim 1, where the Capsaicinoid comprises capsaicin is in the range of 60,000 to 100,000 Scoville Units

4. The topical composition of claim 1, further comprising a safe and effective amount of at least one composition that stimulate production of Collagen and/or Fibril in the skin comprising a palmitoyl oligiopeptide in a weight percent range of 0.010 to 10.000 wt/wt.

5. The topical composition of claim 1, wherein the palmitoyl oligiopeptide comprises palmitoyl pentapeptide-3 and palmitoyl tetrapeptide-3.

6. The topical composition of claim 1, further comprising a safe and effective amount of Lactobacillus/Trifolium Pratense(clover) flower ferment extract & Lactobacillus/Theobroma Cacoa (Cocoa) ferment extract & Lactobacillus/Camellia Senensis leaf ferment extract in a weight percent from 0.010 to 10% wt/wt.

7. The topical composition of claim 1, further comprising at least one of Lactobacillus/Lentinus (Shitake mushroom) ferment extract and lactobacillus/Ganoderma Lucidum(Reishi/Mannentaken mushroom) ferment extract in a weight percent from 0.05 to 10% wt/wt.

8. The topical composition of claim 1, further comprising a Lactobacillus/Theobroma Cacoa (Cocoa) ferment extract in a weight percent from 0.05 to 10% wt/wt.

9. The topical composition of claim 1, further comprising a Lactobacillus lysate extract in a weight percent from 0.05 to 10% wt/wt., which has platelet-derived growth factor (PDGF), insulin-like growth factor (IGF-I, IGF-II), as well as transforming growth factor (TGF-a), which growth factors stimulate fibroblast proliferation as well as the production of extra-cellular proteins such as collagen.

10. A method for reducing sagging or wrinkles in the skin of the neck of humans comprising applying the topical composition of claim 1 to the skin of the neck of a human.

11. A topical composition for reducing sagging or wrinkles in the skin of the neck of humans, comprising:
a safe and effective amount of a composition that stimulates increased localized circulation in the skin comprising a Capsaicinoid in a range of 60,000 to 100,000 Scoville Units in a weight percent range of about 0.4 wt/wt.;
a safe and effective amount of an antispasmodic composition comprising benzaldehyde in a weight percent range of about 2.360 wt/wt. or benzyl cinnamate in a weight percent range of about 0.8 wt/wt.;
a safe and effective amount of skin astringent to tighten the skin comprising camphor in a weight percent range of about 1.6 wt/wt.;
a safe and effective amount of at least one composition that stimulate production of Collagen and/or Fibril in the skin comprising a palmitoyl oligiopeptide in a weight percent range of 0.010 to 10.000 wt/wt.; and
a dermatologically acceptable carrier.

12. The topical composition of claim 11, wherein the Capsaicinoid comprises Capsicum Frutescens Fruit Extract.

13. The topical composition of claim 11, wherein the palmitoyl oligiopeptide comprises palmitoyl pentapeptide-3 and palmitoyl tetrapeptide-3.

14. The topical composition of claim 11, further comprising a safe and effective amount of Lactobacillus/Trifolium Pratense(clover) flower ferment extract & Lactobacillus/Theobroma Cacoa (Cocoa) ferment extract & Lactobacillus/Camellia Senensis leaf ferment extract in a weight percent from 0.010 to 10% wt/wt.

15. The topical composition of claim 11, further comprising a Lactobacillus/Theobroma Cacoa (Cocoa) ferment extract in a weight percent from 0.05 to 10% wt/wt.

16. A method for reducing sagging or wrinkles in the skin of the neck of humans comprising applying the topical composition of claim 11 to the skin of the neck of a human.

17. A topical composition for reducing sagging or wrinkles in the human skin, comprising:
a safe and effective amount of a mild topical astringent to tighten the skin comprising at least one ingredient selected from the group consisting of Camphor, thymol, peppermint oil, menthol and tea tree oil;
a safe and effective amount of a topical antispasmodic to smooth the skin comprising at least one ingredient selected from the group consisting of benzyl cinnamate, benzaldehyde, benzyl nicotinate and benzyl alcohol;
a safe and effective amount of a topical skin irritant comprising at least one ingredient selected from the group consisting of capsaicinoids, oil of black pepper and methyl nicotinate; and
a dermatologically acceptable carrier.

18. The topical composition of claim 17, further comprising a safe and effective amount of palmitoyl pentapeptide-3 to stimulate production of collagen and elastin in skin.

19. A method for reducing sagging or wrinkles in the skin of the neck of humans comprising applying the topical composition of claim 17 to the skin of the neck of a human.
